(19) United States
(12) Patent Application Publication
(10) Pub. No.: US 2021/0361768 A1
(43) Pub. Date: Nov. 25, 2021

(54) METHODS AND MATERIALS FOR ASSESSING AND TREATING CANCERS

(71) Applicant: Mayo Foundation for Medical Education and Research, Rochester, MN (US)

(72) Inventors: Saranya Chumsri, Jacksonville, FL (US); E. Aubrey Thompson, Jacksonville, FL (US)

(21) Appl. No.: 17/049,831

(22) PCT Filed: Jun. 3, 2019
(86) PCT No.: PCT/US2019/035190

§ 371 Date: Oct. 22, 2020

Related U.S. Application Data
(60) Provisional application No. 62/679,431, filed on Jun. 1, 2018.

(51) Int. Cl. A61K 39/395; A61K 31/337; A61K 31/675; A61K 31/704; A61K 31/7068; A61K 31/513; A61P 35/00

(52) U.S. Cl. CPC A61K 39/3955; A61K 31/337; A61K 31/675; A61K 2039/545; A61K 31/7068; A61K 31/513; A61P 35/00; A61K 31/704

(57) ABSTRACT

This document provides methods and materials involved in for assessing and/or treating a mammal having a human epidermal growth factor receptor 2 (HER2)-positive cancer. For example, methods and materials for detecting the presence or absence of an adaptive immune signature and/or a molecular tumor infiltrating lymphocyte signature in a mammal having cancer, thereby identifying that the cancer is likely to respond to a particular cancer treatment, are provided. For example, methods and materials for treating a mammal identified as having a cancer likely to respond to a particular cancer treatment based, at least, in part on the presence or absence of an AIS and/or mTIL are provided.

### CROSS-REFERENCE TO RELATED APPLICATIONS

This application claims priority to U.S. Application Ser. No. 62/679,431, filed on Jun. 1, 2018. The disclosure of the prior application is considered part of the disclosure of this application, and is incorporated in its entirety into this application.

### BACKGROUND

### 1. Technical Field

This document relates to methods and materials involved in assessing and/or treating a mammal having a human epidermal growth factor receptor 2 (HER2)-positive cancer. For example, methods and materials provided herein can be used to detect the adaptive immune signature and/or the molecular tumor infiltrating lymphocyte signature of a mammal having cancer, thereby identifying the cancer as being likely to respond to a particular cancer treatment. For example, methods and materials provided herein can be used to treat a mammal identified as having a cancer likely to respond to a particular cancer treatment.

### 2. Background Information

Trastuzumab is a humanized IgG1 κ monoclonal antibody against extracellular domain of human epidermal growth factor receptor 2 (HER2). Mechanisms of its anti-tumor activities include inhibition of ligand-independent HER2 dimerization, inhibition of downstream signal transduction, induction of cell cycle arrest, induction of apoptosis, inhibition of angiogenesis, and DNA repair interference.

### SUMMARY

This document provides methods and materials for assessing and/or treating a mammal having a HER2-positive cancer. In some cases, this document provides methods and materials for identifying a mammal having a HER2-positive cancer as being responsive to a particular cancer treatment (e.g., by identifying the mammal as having an enriched AIS or a deficient AIS, and, optionally, treating the mammal). For example, this document provides methods and materials for detecting the presence of an adaptive immune signature (AIS) and/or a molecular tumor infiltrating lymphocyte (mTIL) signature of a mammal having cancer (e.g., a HER2-positive cancer). In some cases, an AIS and/or a mTIL signature can detected in a sample from a mammal having cancer. For example, a sample obtained from a mammal having cancer can be assessed to determine if the mammal is likely to be responsive to a particular cancer treatment based, at least in part, on the presence of an AIS and/or a mTIL signature in the sample. As demonstrated herein, a distinct AIS (e.g., an enriched AIS or a deficient AIS) and/or a distinct mTIL signature (e.g., an enriched mTIL signature or a deficient mTIL signature) can indicate that the mammal (e.g., human) is likely to be responsive to a particular cancer treatment (e.g., chemotherapy alone, trastuzumab, lapatinib alone, any combinations thereof, and combinations administered in a particular sequence). For example, mammals (e.g., humans) having an enriched AIS can exhibit improved relapse-free survival (e.g., as compared to patients having a deficient AIS) when treated with chemotherapy and concurrently treated with trastuzumab. In some cases, mammals (e.g., humans) having a deficient AIS can exhibit improved event-free survival (e.g., as compared to patients having an enriched AIS) when treated with lapatinib or treated with a combination of trastuzumab and lapatinib. In some cases, mammals (e.g., humans) having an enriched mTIL signature can exhibit improved relapse-free survival (e.g., as compared to patients having a deficient mTIL signature) when treated with chemotherapy alone. In some cases, mammals (e.g., humans) having an enriched mTIL signature can exhibit improved relapse-free survival (e.g., as compared to patients having a deficient mTIL signature) when treated with chemotherapy and sequentially treated with trastuzumab. In some cases, mammals (e.g., humans) having a deficient mTIL signature can exhibit improved relapse-free survival (e.g., as compared to patients having an enriched mTIL signature) when treated with chemotherapy and concurrently treated with trastuzumab.

Having the ability to identify a particular cancer treatment that a mammal (e.g., a human) is most likely to respond to allows clinicians to provide an individualized approach in selected cancer treatments, thereby improving disease-free survival and/or overall survival and/or minimizing subjecting patients to ineffective treatments.

In general, one aspect of this document features methods for treating a mammal having a HER2-positive cancer and having an enriched AIS. The methods can include, or consist essentially of, administrating trastuzumab to a mammal, and concurrently administering one or more chemotherapeutic agents to that mammal. The enriched AIS can include an elevated level of polypeptides expressed by CD200 receptor 1 (CD200R1), cluster of differentiation (CD) 226 (CD226), TNF Receptor Associated Factor 6 (TRAF6), cytotoxic T-lymphocyte associated protein 4 (CTLA4), CD3-gamma (CD3G), cadherin-1 (CDH1), inducible T-cell co-stimulator (ICOS), integrin alpha L (ITGAL), mitogen-activated protein kinase kinase kinase 8 (MAP3K8), CD28, NFKB inhibitor alpha (NFKBIA), intercellular adhesion molecule 3 (ICAM3), C-terminal Src kinase (CSK), HRAS, SELL, 3-phosphoinositide-dependent protein kinase 1 (PDPK1), and CD3. The enriched AIS can include an elevated level of polypeptides expressed by CD200R1, CD226, TRAF6, CTLA4, CD3G, CDH1, ICOS, ITGAL, MAP3K8, CD28, NFKBIA, ICAM3, CSK, HRAS, SELL, PDPK1, C3, phosphoinositide-3-kinase catalytic delta (PIK3CD), CD3-epsilon (CD3E), 70 kDa zeta-associated protein (ZAP70), FYN, killer cell lectin like receptor K1 (KLRK1), neutrophil cytosolic factor 4 (NCF4), CD3 delta (CD3D), Protein tyrosine phosphatase receptor type C (PTPRC), adhesion molecule interacts with CXADR antigen 1 (AMICA1), CD4, lymphocyte cytosolic protein 2 (LCP2), TNF receptor superfamily member 14 (TNFRSF14), FYN-T-binding protein (FYB), forkhead box protein O1 (FOXO1), HLA class II histocompatibility antigen DM beta (HLA-DMB), CD96, CD74, integrin beta chain-2 (ITGB2), HLA class II histocompatibility antigen DM alpha (HLA-DMA), LCK, Fc fragment of IgG receptor IIb (FCGR2B), and hematopoietic cell signal transducer (HCST). The administering one or more chemotherapeutic agents to the mammal can include first administering 3 cycles of docetaxel and capecitabine, and subsequently administering 3 cycles of cyclophosphamide, epirubicin, and capecitabine. The administering one or more chemotherapeutic agents to the mammal can include first administering 3 cycles of docetaxel, and subsequently administering by 3 cycles of cyclophosphamide, epirubicin, and fluorouracil. The mammal can be a human. The cancer can be a breast cancer. The breast cancer can be a hormone receptor positive breast cancer.

In another aspect, this document features methods for treating a mammal having a HER2-positive cancer and having a deficient AIS. The methods can include, or consist essentially of, administering trastuzumab to a mammal, and sequentially administering one or more chemotherapeutic agents to that mammal. The administering one or more chemotherapeutic agents to the mammal can include first administering 3 cycles of docetaxel and capecitabine, and subsequently administering 3 cycles of cyclophosphamide, epirubicin, and capecitabine. The administering one or more chemotherapeutic agents to the mammal can include first administering 3 cycles of docetaxel, and subsequently administering by 3 cycles of cyclophosphamide, epirubicin, and fluorouracil. The sequentially administering the one or more chemotherapeutic agents can include administering the one or more chemotherapeutic agents after administering the trastuzumab. The sequentially administering the one or more chemotherapeutic agents can include administering the one or more chemotherapeutic agents before administering the trastuzumab. The deficient AIS can include a reduced level of polypeptides expressed by CD200R1, CD226, TRAF6, CTLA4, CD3G, CDH1, ICOS, ITGAL, MAP3K8, CD28, NFKBIA, ICAM3, CSK, HRAS, SELL, PDPK1, and CD3. The deficient can include a reduced level of polypeptides expressed by CD200R1, CD226, TRAF6, CTLA4, CD3G, CDH1, ICOS, ITGAL, MAP3K8, CD28, NFKBIA, ICAM3, CSK, HRAS, SELL, PDPK1, C3, PIK3CD, CD3E, ZAP70, FYN, KLRK1, NCF4, CD3D, PTPRC, AMICA1, CD4, LCP2, TNFRSF14, FYB, FOXO1, HLA-DMB, CD96, CD74, ITGB2, HLA-DMA, LCK, FCGR2B, and HCST. The mammal can be a human. The cancer can be a breast cancer. The breast cancer can be a hormone receptor positive breast cancer.

In another aspect, this document features methods for treating a mammal having a HER2-positive cancer and having a deficient AIS. The methods can include, or consist essentially of, administering trastuzumab to a mammal, and administering lapatinib, neratinib, and/or afatinib to that mammal. The deficient AIS can include a reduced level of polypeptides expressed by CD200R1, CD226, TRAF6, CTLA4, CD3G, CDH1, ICOS, ITGAL, MAP3K8, CD28, NFKBIA, ICAM3, CSK, HRAS, SELL, PDPK1, and CD3. The deficient can include a reduced level of polypeptides expressed by CD200R1, CD226, TRAF6, CTLA4, CD3G, CDH1, ICOS, ITGAL, MAP3K8, CD28, NFKBIA, ICAM3, CSK, HRAS, SELL, PDPK1, C3, PIK3CD, CD3E, ZAP70, FYN, KLRK1, NCF4, CD3D, PTPRC, AMICA1, CD4, LCP2, TNFRSF14, FYB, FOXO1, HLA-DMB, CD96, CD74, ITGB2, HLA-DMA, LCK, FCGR2B, and HCST. The mammal can be a human. The cancer can be a breast cancer. The breast cancer can be a hormone receptor positive breast cancer.

In another aspect, this document features methods for treating a mammal having a HER2-positive cancer and having a deficient AIS. The methods can include, or consist essentially of, administering lapatinib, neratinib, and/or afatinib to a mammal. The deficient AIS can include a reduced level of polypeptides expressed by CD200R1, CD226, TRAF6, CTLA4, CD3G, CDH1, ICOS, ITGAL, MAP3K8, CD28, NFKBIA, ICAM3, CSK, HRAS, SELL, PDPK1, and CD3. The deficient can include a reduced level of polypeptides expressed by CD200R1, CD226, TRAF6, CTLA4, CD3G, CDH1, ICOS, ITGAL, MAP3K8, CD28, NFKBIA, ICAM3, CSK, HRAS, SELL, PDPK1, C3, PIK3CD, CD3E, ZAP70, FYN, KLRK1, NCF4, CD3D, PTPRC, AMICA1, CD4, LCP2, TNFRSF14, FYB, FOXO1, HLA-DMB, CD96, CD74, ITGB2, HLA-DMA, LCK, FCGR2B, and HCST. The mammal can be a human. The cancer can be a breast cancer. The breast cancer can be a hormone receptor positive breast cancer.

In another aspect, this document features methods for treating a mammal having a HER2-positive cancer and having an enriched mTIL signature. The methods can include, or consist essentially of, administering trastuzumab to a mammal, and sequentially administering one or more chemotherapeutic agents to that mammal. The enriched mTIL signature can include an increased abundance of B cells, CD8 T cells, cytotoxic cells, exhausted CD8, immature dendritic cells (iDC), macrophages, mast cells, neutrophils, NK CD56 dim cells, T cells, and regulatory T cells (Treg). The administering one or more chemotherapeutic agents to the mammal can include first administering 3 cycles of docetaxel and capecitabine, and subsequently administering 3 cycles of cyclophosphamide, epirubicin, and capecitabine. The administering one or more chemotherapeutic agents to the mammal can include first administering 3 cycles of docetaxel, and subsequently administering by 3 cycles of cyclophosphamide, epirubicin, and fluorouracil. The sequentially administering the one or more chemotherapeutic agents to the mammal can include administering the one or more chemotherapeutic agents after administering the trastuzumab. The sequentially administering said one or more chemotherapeutic agents to the mammal can include administering the one or more chemotherapeutic agents before administering the trastuzumab. The mammal can be a human. The cancer can be a breast cancer. The breast cancer can be a hormone receptor positive breast cancer.

In another aspect, this document features methods for treating a mammal having a HER2-positive cancer and having a deficient mTIL signature. The methods can include, or consist essentially of, administrating trastuzumab to a mammal, and concurrently administering one or more chemotherapeutic agents to that mammal. The deficient mTIL signature can include a reduced abundance of B cells, CD8 T cells, cytotoxic cells, exhausted CD8, iDCs, macrophages, mast cells, neutrophils, NK CD56 dim cells, T cells, and Tregs. The administering one or more chemotherapeutic agents to the mammal can include first administering 3 cycles of docetaxel and capecitabine, and subsequently administering 3 cycles of cyclophosphamide, epirubicin, and capecitabine. The administering one or more chemotherapeutic agents to the mammal can include first administering 3 cycles of docetaxel, and subsequently administering by 3 cycles of cyclophosphamide, epirubicin, and fluorouracil. The mammal can be a human. The cancer can be a breast cancer. The breast cancer can be a hormone receptor positive breast cancer.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C contain Kaplan Meier curves of recurrence-free survival (RFS) in the NCCTG N9831 comparing between patients with enriched vs. deficient CD45 mTIL signature. FIG. 1A shows a Kaplan Meier curve for patients treated with a chemotherapy only arm (AC-T). FIG. 1B shows a Kaplan Meier curve for patients treated with a sequential trastuzumab arm (AC-T-H); FIG. 1C shows a Kaplan Meier curve for patients treated with a concurrent trastuzumab arm (AC-TH).

FIGS. 2A-2C contain Forest plots of RFS with immune subset signatures in the NCCTG N9831. FIG. 2A shows a Forest plot of patients treated with a chemotherapy only arm (AC-T). FIG. 2B shows a Forest plot of patients treated with a sequential trastuzumab arm (AC-T-H). FIG. 2C shows a Forest plot of patients treated with a concurrent trastuzumab arm (AC-TH).

FIGS. 3A-3B contain Forest plots of RFS with immune subset signatures in the combined FinHER and FinXX trials. FIG. 3A shows a Forest plot of patients who received chemotherapy alone. FIG. 3B shows a Forest plot of patients who received chemotherapy in combination with trastuzumab.

FIG. 4 contains results showing survival of patients in the NCCTG N9831. A Forest plot of RFS in patients with adaptive immune signature in the NCCTG N9831 treated with chemotherapy alone (AC-T), chemotherapy followed by sequential trastuzumab (AC-T-H), or chemotherapy concurrent with trastuzumab (AC-TH). 14-year event free survival (EFS) in the NCCTG N9831 trial based on treatment arms and adaptive immune signature status (enriched vs. deficient) is shown in the table.

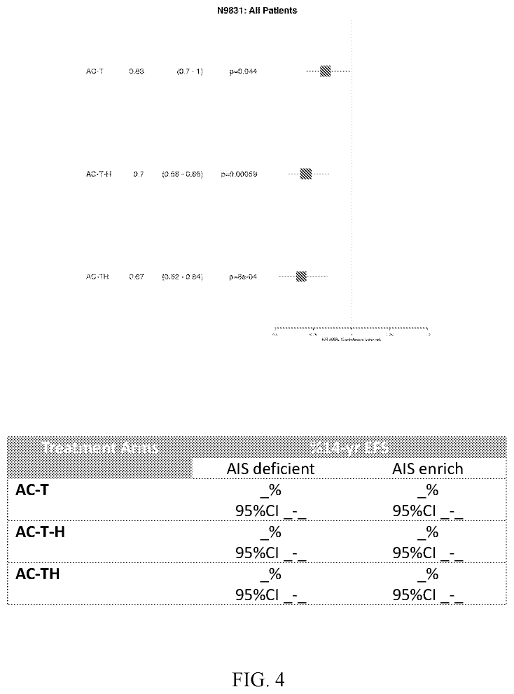

FIGS. 5A-5C contains Kaplan Meier curves of RFS in patients with deficient adaptive immune signature in the NeoALTTO. FIG. 5A shows patients treated with lapatinib. FIG. 5B shows patients treated with trastuzumab. FIG. 5C shows patients treated with a combination of lapatinib and trastuzumab (lap/tras)).

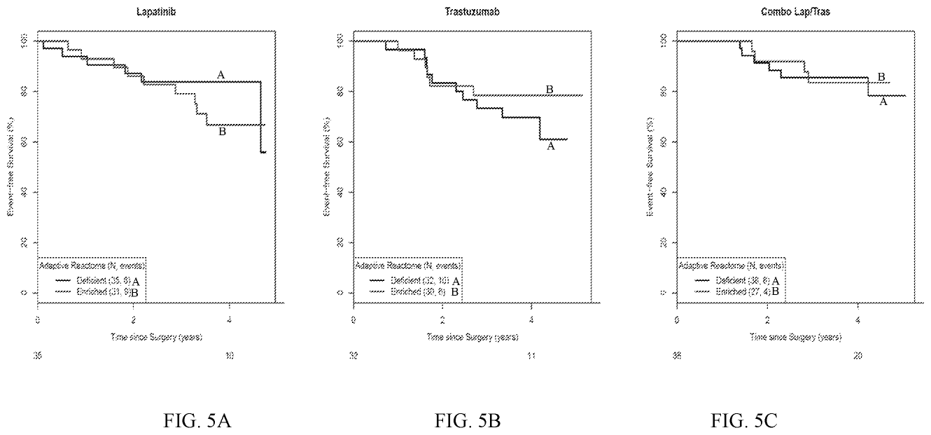

FIG. 6 contains an adaptive immune signature and pathological quantification of a stromal tumor infiltrating lymphocyte (TIL) population.

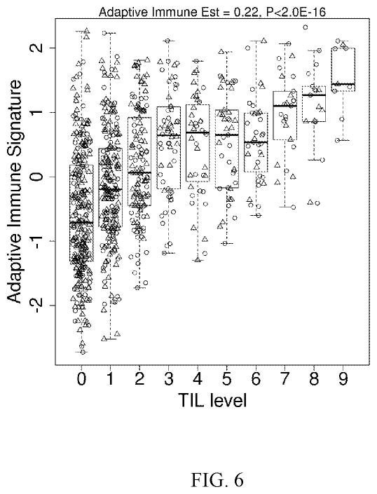

### DETALED DESCIRPTION

This document provides methods and materials for assessing and/or treating a mammal having a HER2-positive cancer. For example, this document provides methods and materials for identifying a mammal having a HER2-positive cancer as being responsive to a particular cancer treatment (e.g., by detecting the AIS and/or the mTIL signature), and, optionally, treating the mammal. In some cases, the methods and materials described herein can be used to predict response to a particular cancer treatment. For example, a sample obtained from a mammal (e.g., a human) having, or suspected of having, a HER2-positive cancer can be assessed to determine if the mammal is likely to be responsive to a particular cancer treatment (e.g., chemotherapy alone, trastuzumab, lapatinib alone, any combinations thereof, and combinations administered in a particular sequence) based, at least in part, on an AIS and/or a mTIL signature present in the sample.

In some cases, the methods and materials described herein can be used to treat a mammal having, or suspected of having, a HER2-positive cancer. For example, a mammal having, or suspected of having, a HER2-positive cancer identified as being likely to be responsive to a particular cancer treatment based, at least in part, on an AIS and/or a mTIL signature present in the sample can be treated with that particular cancer treatment as described herein. In some cases, the methods and materials described herein can be used to improve disease-free (e.g., relapse-free survival. In some cases, the methods and materials described herein can be used to improve overall survival.

When treating a mammal having, or suspected of having, a HER2-positive cancer as described herein, the treatment can be effective to treat the cancer (e.g., to reduce one or more symptoms of the cancer). In some cases, the number of cancer cells present within a mammal can be reduced using the materials and methods described herein. In some cases, the size (e.g., volume) of one or more tumors present within a mammal can be reduced using the materials and methods described herein. In some cases, the size (e.g., volume) of one or more tumors present within a mammal does not increase.

Any type of mammal having, or suspected of having, a HER2-positive cancer can be assessed and/or treated as described herein. Examples of mammals that can be assessed and/or treated as described herein include, without limitation, primates (e.g., humans and monkeys), dogs, cats, horses, cows, pigs, sheep, rabbits, mice, and rats. For example, the mammal can be a human. In some cases, a human having a HER2-positive cancer can be assessed to determine if the mammal is likely to be responsive to a particular cancer treatment based, at least in part, on an AIS present in the sample and/or treated with a particular cancer treatment as described herein.

A mammal having, or suspected of having, any type of HER2-positive cancer can be assessed and/or treated as described herein. Examples of HER2-positive cancers that can be assessed and/or treated as described herein include, without limitation, breast cancers, prostate cancers, ovarian cancers, gastric cancers such as gastroesophageal cancers, endometrial cancers, bladder cancers, lung cancers, colon cancers, and head and neck cancers. A HER2-positive cancer can be a hormone receptor positive cancer or a hormone receptor negative cancer. For example, HER2-positive cancer can be hormone receptor positive breast cancer. For example, HER2-positive cancer can be hormone receptor positive breast cancer. Examples of hormone receptors include, without limitation, estrogen receptors, androgen receptors, follicle-stimulating hormone receptors, growth hormone receptors, luteinizing hormone receptors, and progesterone receptors.

In some cases, a mammal can be identified as having a HER2-positive cancer. Any appropriate method can be used to identify a mammal as having a HER2-postive cancer. For example, imaging techniques and biopsy techniques can be used to identify mammals (e.g., humans) having cancer. For example, the presence, absence, or level of HER2 polypeptides can be detected in a sample (e.g., a tumor sample such as a cancer biopsy) obtained from a mammal to determine if the mammal has a HER2-positive cancer. For example, when the presence of HER2 polypeptides is detected, the mammal can be identified as having a HER2-positive cancer.

A mammal having, or suspected of having, a HER2-positive cancer, can be assessed to determine whether or not it is likely to respond to a particular cancer treatment (e.g., chemotherapy alone, trastuzumab, lapatinib alone, any combinations thereof, and combinations administered in a particular sequence). For example, a sample obtained from the mammal can be assessed for an AIS and/or a mTIL signature as described herein, and the AIS and/or the mTIL signature of that mammal can be used to determine whether or not that mammal is likely to respond to a particular cancer treatment.

Any appropriate sample from a mammal (e.g., a human) having, or suspected of having, a HER2-positive cancer can be assessed as described herein. In some cases, a sample can be a biological sample. Examples of biological samples include, without limitation, tissue samples (e.g., tumor, cancer, heart, lung, gastrointestinal tract, kidney, ovary, uterus, lung, or head and neck tissues) and fluid samples (e.g., blood, serum, plasma, or urine). A biological sample can be a fresh sample or a fixed sample. In some cases, a biological sample can be a processed (e.g., paraffin embedded) sample. For example, a biological sample from a mammal having a HER2-positive cancer can be a paraffin embedded tumor samples.

An AIS can be any appropriate AIS. For example, an AIS can include the presence, absence, or level of polypeptides expressed by one or more immune related genes in a sample from a mammal having a HER2-positive cancer. An AIS can include two or more (e.g., three, four, five, six, seven, eight, nine, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or more) polypeptides expressed by immune related genes. Examples of polypeptides expressed by immune related genes that can be included in an AIS described herein include, without limitation, CD200R1, CD226, TRAF6, CTLA4, CD3G, CDH1, ICOS, ITGAL, MAP3K8, CD28, NFKBIA, ICAM3, CSK, HRAS, SELL, PDPK1, C3, PIK3CD, CD3E, ZAP70, FYN, KLRK1, NCF4, CD3D, PTPRC, AMICA1, CD4, LCP2, TNFRSF14, FYB, FOXO1, HLA-DMB, CD96, CD74, ITGB2, HLA-DMA, LCK, FCGR2B, and HCST. In some cases, an AIS can include 17 polypeptides expressed by immune related genes (e.g., CD200R1, CD226, TRAF6, CTLA4, CD3G, CDH1, ICOS, ITGAL, MAP3K8, CD28, NFKBIA, ICAM3, CSK, HRAS, SELL, PDPK1, and CD3). In some cases, an AIS can include polypeptides expressed by the immune related genes set forth in Table 3. For example, an AIS can include polypeptides expressed by CD200R1, CD226, TRAF6, CTLA4, CD3G, CDH1, ICOS, ITGAL, MAP3K8, CD28, NFKBIA, ICAM3, CSK, HRAS, SELL, PDPK1, C3, PIK3CD, CD3E, ZAP70, FYN, KLRK1, NCF4, CD3D, PTPRC, AMICA1, CD4, LCP2, TNFRSF14, FYB, FOXO1, HLA-DMB, CD96, CD74, ITGB2, HLA-DMA, LCK, FCGR2B, and HCST. In some cases, an AIS can include 39 polypeptides expressed by immune related genes. For example, determining if a mammal having, or suspected of having, a HER2-positive cancer is likely to be responsive to a particular cancer treatment can be based, at least in part, on the AIS of that mammal.

In some cases, an AIS can be an enriched AIS. An enriched AIS can include the presence and/or an elevated level of one or more polypeptides expressed by immune related genes. For example, an enriched AIS can include an elevated level of one or more polypeptides expressed by CD200R1, CD226, TRAF6, CTLA4, CD3G, CDH1, ICOS, ITGAL, MAP3K8, CD28, NFKBIA, ICAM3, CSK, HRAS, SELL, PDPK1, C3, PIK3CD, CD3E, ZAP70, FYN, KLRK1, NCF4, CD3D, PTPRC, AMICA1, CD4, LCP2, TNFRSF14, FYB, FOXO1, HLA-DMB, CD96, CD74, ITGB2, HLA-DMA, LCK, FCGR2B, and HCST. The term “elevated level” as used herein with respect to a level of one or more polypeptides expressed by immune related genes refers to any level that is greater than a reference level of one or more respective polypeptides expressed by immune related genes. The term “reference level” as used herein with respect to one or more polypeptides expressed by immune related genes refers to the level of one or more polypeptides expressed by immune related genes typically observed in a sample (e.g., a control sample) from one or more mammals (e.g., humans) without cancer. Control samples can include, without limitation, samples from normal (e.g., healthy) mammals, and cell lines (e.g., non-tumor forming cells lines). In some cases, an enriched AIS can indicate that a mammal is likely to have an increased stromal TIL (sTIL) population. In some cases, an enriched AIS can indicate that a mammal is likely to have a hormone receptor (e.g., an estrogen receptor) negative cancer. In some cases, an enriched AIS can indicate that a mammal is likely to have pathologic complete response (pCR), respond to, or have better survival when treated with one or more agents that inhibit HER2 activity (e.g., one or more anti-HER2 antibodies such as trastuzumab and pertuzumab).

In some cases, an AIS can be a deficient AIS. A deficient AIS can include the absence and/or a reduced level of one or more polypeptides expressed by immune related genes. For example, a deficient AIS can include a reduced level of one or more polypeptides expressed by CD200R1, CD226, TRAF6, CTLA4, CD3G, CDH1, ICOS, ITGAL, MAP3K8, CD28, NFKBIA, ICAM3, CSK, HRAS, SELL, PDPK1, C3, PIK3CD, CD3E, ZAP70, FYN, KLRK1, NCF4, CD3D, PTPRC, AMICA1, CD4, LCP2, TNFRSF14, FYB, FOXO1, HLA-DMB, CD96, CD74, ITGB2, HLA-DMA, LCK, FCGR2B, and HCST. The term “reduced level” as used herein with respect to a level of one or more polypeptides expressed by immune related genes refers to any level that is lower than a reference level of one or more respective polypeptides expressed by immune related genes. The term “reference level” as used herein with respect to one or more polypeptides expressed by immune related genes refers to the level of one or more polypeptides expressed by immune related genes typically observed in a sample (e.g., a control sample) from one or more mammals (e.g., humans) without cancer. Control samples can include, without limitation, samples from normal (e.g., healthy) mammals, and cell lines (e.g., non-tumor forming cells lines). In some cases, a deficient AIS can indicate that a mammal is less likely to have pathologic complete response (pCR), respond to, or have better survival with monoclonal antibodies against HER2, such as, but not limited to, trastuzumab and pertuzumab. In some cases, a deficient AIS can indicate that a mammal is more likely to have pathologic complete response (pCR), respond to, or have better survival when treated with one or more agents that inhibit HER2 activity (e.g., one or more small molecule inhibitors of HER2 such aslapatinib, neratinib, and tucatinib).

A mTIL signature can be any appropriate mTIL signature. For example, a mTIL signature can include the presence, absence, or abundance of one or more immune cell populations in a sample from a mammal having a HER2-positive cancer. In some cases, a mTIL signature can include one or more (e.g., one, two, three, four, five, six, seven, eight, nine, 10, 11, 12, 13, 14, or more) immune cell populations. Examples of immune cell populations that can be included in a mTIL signature described herein include, without limitation, CD45 cells, B cells, CD8 T cells, cytotoxic cells, exhausted CD8, immature dendritic cells (iDCs), macrophages, mast cells, neutrophils, NK CD56 dim cells, T cells, and regulatory T cells (Tregs). For example, a mTIL signature can include one immune cell population (e.g., an immune subset signature). For example, a mTIL signature can include 11 immune cell populations (e.g., a total mTIL signature). In some cases, a total mTIL signature can include CD45 cells B cells, CD8 T cells, cytotoxic cells, exhausted CD8, iDCs, macrophages, mast cells, neutrophils, NK CD56 dim cells, T cells, and Tregs. For example, determining if a mammal having, or suspected of having, a HER2-positive cancer is likely to be responsive to a particular cancer treatment can be based, at least in part, on the mTIL signature of that mammal.

In some cases, a mTIL signature can be an enriched mTIL signature. An enriched mTIL signature can include the presence and/or increased abundance of one or more immune cell populations. For example, an enriched mTIL signature can include an increased abundance of one or more of CD45 cells, B cells, CD8 T cells, cytotoxic cells, exhausted CD8, iDCs, macrophages, mast cells, neutrophils, NK CD56 dim cells, T cells, and Tregs. The term “increased abundance” as used herein with respect to a level of one or more immune cell populations refers to any level that is greater than a reference level of one or more respective immune cell populations. The term “reference level” as used herein with respect to one or more immune cell populations refers to the level of one or more immune cell populations typically observed in a sample (e.g., a control sample) from one or more mammals (e.g., humans) without cancer. Control samples can include, without limitation, samples from normal (e.g., healthy) mammals, and cell lines (e.g., non-tumor forming cells lines). For example, an enriched mTIL signature can include an increased abundance B cells, CD8 T cells, cytotoxic cells, exhausted CD8, iDCs, macrophages, mast cells, neutrophils, NK CD56 dim cells, T cells, and Tregs.

In some cases, a mTIL signature can be a deficient mTIL signature. A deficient mTIL signature can include the absence and/or a reduced amount of one or more immune cell populations. For example, an enriched mTIL signature can include an increased abundance of one or more of CD45 cells, B cells, CD8 T cells, cytotoxic cells, exhausted CD8, iDCs, macrophages, mast cells, neutrophils, NK CD56 dim cells, T cells, and Tregs. The term “reduced abundance” as used herein with respect to a level of one or more immune cell populations refers to any level that is greater than a reference level of one or more respective immune cell populations. The term “reference level” as used herein with respect to one or more immune cell populations refers to the level of one or more immune cell populations typically observed in a sample (e.g., a control sample) from one or more mammals (e.g., humans) without cancer. Control samples can include, without limitation, samples from normal (e.g., healthy) mammals, and cell lines (e.g., non-tumor forming cells lines). For example, a deficient mTIL signature can include a reduced abundance CD45 cells, B cells, CD8 T cells, cytotoxic cells, exhausted CD8, iDCs, macrophages, mast cells, neutrophils, NK CD56 dim cells, T cells, and Tregs.

Any appropriate method can be used to detect the AIS and/or the mTIL signature of a mammal having, or suspected of having, a HER2-positive cancer. In some cases, when detecting the AIS, any appropriate method can be used to determine the presence, absence, or level of one or more polypeptides expressed by immune related genes within a sample. For example, the presence, absence, or level of one or more polypeptides expressed by immune related genes can be assessed by detecting and/or quantifying mRNA of immune related genes. In some cases, when detecting the mTIL signature, any appropriate method can be used to determine the presence, absence, or abundance of one or more immune cell populations within a sample. For example, the presence, absence, or abundance of one or more immune cell populations can be assessed by detecting and/or quantifying mRNA of those marker genes. For example, a total mTIL can be assessed by detecting and/or quantifying CD45. For example, a mTIL immune subset signature can be assessed by detecting and/or quantifying mRNA of one or more marker genes as set forth in Table 3. Examples of methods that can be used to detect the polypeptides and mRNA include, without limitation, immunohistochemistry (IHC) techniques, mass spectrometry techniques (e.g., proteomics-based mass spectrometry assays or targeted quantification-based mass spectrometry assays), western blotting techniques, quantitative RT-PCR techniques, and multiplexed digital barcoding multiplexed techniques. In some cases, an AIS and/or a mTIL signature can be assessed by quantifying mRNA in a sample from a mammal having a HER2-positive cancer. In some cases, an AIS and/or a mTIL signature can be assessed as described elsewhere (see, e.g., Perez et al., 2015 J. Clin. Oncol., 33:701-8; and Danaher et al., 2017 J. Immunother. Cancer, 5:18).

A mammal having, or suspected of having, a HER2-positive cancer can be administered, or instructed to self-administer, one or more cancer treatments. For example, an AIS and/or a mTIL signature of a mammal having, or suspected of having, a HER2-positive cancer can be used to determine the responsiveness of that mammal to a particular cancer treatment, and a treatment option for the mammal (e.g., an individualized cancer treatment) can be selected. Individualized cancer treatments for the treatment of a mammal having, or suspected of having, a HER2-postive cancer (e.g., based on an AIS and/or a mTIL signature as described herein) can include any one or more (e.g., 1, 2, 3, 4, 5, 6, or more) cancer treatments. A cancer treatment can include any appropriate cancer treatment. In some cases, a cancer treatment can include administering one or more chemotherapeutic agents. Examples of chemotherapeutic agents that can be included in a cancer treatment described herein include, without limitation, anthracycline (e.g., doxorubicin), vinorelbine, docetaxel, capecitabine, cyclophosphamide, epirubicin, fluorouracil, carboplatin, paclitaxel, and combinations thereof (e.g., doxorubicin+cyclophosphamide+paclitaxel+carboplatin or doxorubicin+cyclophosphamide+docetaxel+carboplatin). For example, administering one or more chemotherapeutic agents to a mammal having a HER2-positive cancer can include administering 3 cycles of docetaxel and capecitabine followed by 3 cycles of cyclophosphamide, epirubicin, and capecitabine (TX/CEX). For example, administering one or more chemotherapeutic agents to a mammal having a HER2-positive cancer can include administering 3 cycles of docetaxel followed by 3 cycles of cyclophosphamide, epirubicin, and fluorouracil (T/CEF). In some cases, a cancer treatment can include administering one or more agents that inhibit HER2 activity. An agent that can inhibit HER2 activity can be any appropriate type of agent (e.g., an antibody such as a monoclonal antibody or a small molecule). In some cases, an agent that inhibits HER2 activity can inhibit ligand-independent HER2 dimerization and/or inhibit signal transduction downstream of HER2. Examples of agents that inhibit HER2 activity include, without limitation, trastuzumab, pertuzumab, margetuximab, and ado-trastuzumab emtansine. An agent that inhibits HER2 activity also can inhibit one or more additional receptor tyrosine kinases (e.g., other than HER2). For example, the additional receptor tyrosine kinase can be an epidermal growth factor receptor (EGFR), human epidermal growth factor receptor 3 (HER3), or human epidermal growth factor receptor 4 (HER4). Examples of agents that can inhibit HER2 activity and also can inhibit one or more additional receptor tyrosine kinases (e.g., EGFR) include, without limitation, lapatinib, neratinib, afatinib, and tucatinib. In some cases, a cancer treatment can include surgery. In some cases, a cancer treatment can include radiation treatment. In some cases, a cancer treatment for a mammal having, or suspected of having, a HER2-postive cancer can be selected based on the mammal's AIS as described herein. For example, an AIS of mammal having, or suspected of having, a HER2-positive cancer can be used to select a treatment option as described in the Examples and/or as set forth in Table 1.

TABLE 1
Treatment options.

| | Exemplary Treatment Option(s) |
|---|---|
| enriched AIS | chemotherapy + trastuzumab |
| | chemotherapy + trastuzumab + |
| | pertuzumab |
| | trastuzumab |
| | trastuzumab + pertuzumab |
| | Ado-trastuzumab emtansine |
| deficient AIS | lapatinib |
| | neratinib |
| | tucatinib |
| | sequential neratinib after completion |
| | of trastuzumab |
| | sequential tucatinib after completion |
| | of trastuzumab |
| | trastuzumab + lapatinib |
| | trastuzumab + neratinib |
| | trastuzumab + tucatinib |
| | chemotherapy + lapatinib |
| | chemotherapy + neratinib |
| | chemotherapy + tucatinib |
| | chemotherapy + trastuzumab + |
| | lapatinib |
| | chemotherapy + trastuzumab + |
| | neratinib |
| | chemotherapy + trastuzumab + |
| | tucatinib |

In some cases, a cancer treatment for a mammal having, or suspected of having, a HER2-postive cancer can be selected based on the mammal's mTIL signature as described herein. For example, a mTIL of mammal having, or suspected of having, a HER2-positive cancer can be used to select a treatment option as described in the Examples and/or as set forth in Table 2.

TABLE 2
Treatment options.

| | Exemplary Treatment Option(s) |
|---|---|
| enriched mTIL | chemotherapy + trastuzumab |
| | chemotherapy + trastuzumab + |
| | pertuzumab |
| | trastuzumab |
| | trastuzumab + pertuzumab |
| | Ado-trastuzumab emtansine |
| deficient mTIL | lapatinib |
| | neratinib |
| | tucatinib |
| | sequential neratinib after completion |
| | of trastuzumab |
| | sequential tucatinib after completion |
| | of trastuzumab |
| | trastuzumab + lapatinib |
| | trastuzumab + neratinib |
| | trastuzumab + tucatinib |
| | chemotherapy + lapatinib |
| | chemotherapy + neratinib |
| | chemotherapy + tucatinib |
| | chemotherapy + trastuzumab + |
| | lapatinib |
| | chemotherapy + trastuzumab + |
| | neratinib |
| | chemotherapy + trastuzumab + |
| | tucatinib |

In some cases, when treating a mammal (e.g., human) having a HER2-postive cancer and identified as having an enriched AIS as described herein, the mammal can be administered, or instructed to self-administer, one or more chemotherapeutic agents (e.g., anthracycline, vinorelbine, docetaxel, capecitabine, cyclophosphamide, epirubicin, fluorouracil, paclitaxel, and combinations thereof) and can be administered, or instructed to self-administer, one or more agents that inhibit HER2 activity (e.g., trastuzumab). For example, a human having a HER2-positive cancer and an enriched AIS can be administered one or more of anthracycline, vinorelbine, docetaxel, capecitabine, cyclophosphamide, epirubicin, fluorouracil, paclitaxel, and combinations thereof (e.g., TX/CEX and T/CEF), and can be concurrently administered trastuzumab.

In some cases, when treating a mammal (e.g., human) having a HER2-postive cancer and identified as having a deficient AIS as described herein, the mammal can be administered, or instructed to self-administer, one or more chemotherapeutic agents (e.g., anthracycline, vinorelbine, docetaxel, capecitabine, cyclophosphamide, epirubicin, fluorouracil, paclitaxel, and combinations thereof) and can be administered, or instructed to self-administer, one or more agents that inhibit HER2 activity (e.g., trastuzumab). For example, a human having a HER2-positive cancer and a deficient AIS can be administered one or more of anthracycline, vinorelbine, docetaxel, capecitabine, cyclophosphamide, epirubicin, fluorouracil, paclitaxel, and combinations thereof (e.g., TX/CEX and T/CEF), and can be sequentially administered (e.g., before and/or after) trastuzumab.

In some cases, when treating a mammal (e.g., human) having a HER2-postive cancer and identified as having a deficient AIS as described herein, the mammal can be administered, or instructed to self-administer, one or more agents that inhibit HER2 activity (e.g., trastuzumab) and can be administered, or instructed to self-administer, one or more agents that inhibit HER2 activity and inhibit one or more additional receptor tyrosine kinases (e.g., EGFR). For example, a human having a HER2-positive cancer and a deficient AIS can be administered trastuzumab, and can be administered lapatinib.

In some cases, when treating a mammal (e.g., human) having a HER2-postive cancer and identified as having a deficient AIS as described herein, the mammal can be administered, or instructed to self-administer, one or more agents that inhibit inhibit one or more additional receptor tyrosine kinases other than HER2 (e.g., EGFR). For example, a human having a HER2-positive cancer and a deficient AIS can be administered lapatinib.

In some cases, when treating a mammal (e.g., human) having a HER2-postive cancer and identified as having an enriched mTIL as described herein, the mammal can be administered, or instructed to self-administer, one or more chemotherapeutic agents (e.g., anthracycline, vinorelbine, docetaxel, capecitabine, cyclophosphamide, epirubicin, fluorouracil, paclitaxel, and combinations thereof) and can be administered, or instructed to self-administer, one or more agents that inhibit HER2 activity (e.g., trastuzumab). For example, a human having a HER2-positive cancer and an enriched total mTIL can be administered one or more of anthracycline, vinorelbine, docetaxel, capecitabine, cyclophosphamide, epirubicin, fluorouracil, paclitaxel, and combinations thereof (e.g., TX/CEX and T/CEF), and can be sequentially administered (e.g., before and/or after) trastuzumab.

In some cases, when treating a mammal (e.g., human) having a HER2-postive cancer and identified as having an enriched mTIL subset as described herein, the mammal can be administered, or instructed to self-administer, one or more chemotherapeutic agents (e.g., anthracycline, vinorelbine, docetaxel, capecitabine, cyclophosphamide, epirubicin, fluorouracil, paclitaxel, and combinations thereof). For example, a human having a HER2-positive cancer and an enriched mTIL subset (e.g., B-cell, CD8-T-cells, cytotoxic-cell, T-cells, and regulatory-T-cell) can be administered one or more of anthracycline, vinorelbine, docetaxel, capecitabine, cyclophosphamide, epirubicin, fluorouracil, paclitaxel, and combinations thereof (e.g., TX/CEX and T/CEF).

In some cases, when treating a mammal (e.g., human) having a HER2-postive cancer and identified as having a deficient mTIL as described herein, the mammal can be administered, or instructed to self-administer, one or more chemotherapeutic agents (e.g., anthracycline, vinorelbine, docetaxel, capecitabine, cyclophosphamide, epirubicin, fluorouracil, paclitaxel, and combinations thereof) and can be administered, or instructed to self-administer, one or more agents that inhibit HER2 activity (e.g., trastuzumab). For example, a human having a HER2-positive cancer and a deficient total mTIL can be administered one or more of anthracycline, vinorelbine, docetaxel, capecitabine, cyclophosphamide, epirubicin, fluorouracil, paclitaxel, and combinations thereof (e.g., TX/CEX and T/CEF), and can be concurrently administered trastuzumab.

### EXAMPLES

### Example 1

### Association Between Adaptive Immune Signature and Outcome in HER2-Positve Breast Cancer Treated with Trastuzumab and Lapatinib

### Materials and Methods

### Patient Population

A total of 1,280 patients from the NCCTG N9831 (Perez EA et al., Journal of clinical oncology: official journal of the American Society of Clinical Oncology, 32:3744-52 (2014)), 168 patients in FinHer (Joensuu H et al., N Engl. J. Med, 354:809-20 (2006)), and 170 patients in FinXX (Joensuu H et al., Journal of clinical oncology: official journal of the American Society of Clinical Oncology, 30:11-8 (2012)), were included in this analysis. In the NCCTG N9831, patients in arm A were treated with chemotherapy alone (AC-T), arm B received chemotherapy followed by sequential trastuzumab (AC-T-H), and arm C received trastuzumab concurrently with chemotherapy (AC-TH). In the FinHer trial (Joensuu H et al., N. Engl. J. Med., 354:809-20 (2006)), patients were randomized to receive either docetaxel or vinorelbine with or without trastuzumab for 9 weeks. In the FinXX trial (Joensuu H et al., Journal of clinical oncology: official journal of the American Society of Clinical Oncology, 30:11-8 (2012)), patients were randomized to receive either 3 cycles of docetaxel and capecitabine followed by 3 cycles of cyclophosphamide, epirubicin, and capecitabine (TX/CEX) or 3 cycles of docetaxel followed by 3 cycles of cyclophosphamide, epirubicin, and fluorouracil (T/CEF). After May 2005, concurrent administration of trastuzumab was allowed; 96 (13%) and 83 patients (11%) assigned to TX/CEX and T/CEF, respectively, received trastuzumab for either 9 weeks or up to 1 year. Median follow-up was 10.6 years (range 0.8 months-15.3 years) in the NCCTG N9831 trial, 5.5 years in the FinHER trial, and 7.0 years in the FinXX trial.

Molecular TIL (mTIL) and Immune Subset Signatures

NanoString™ technology was used to quantify mRNA in paraffin embedded tumor samples from the NCCTG N9831, FinHER, and FinXX trials. Immune subset signatures were calculated using normalized and log2 transformed data based on previous publication by Danaher et al. (Danaher P et al., J Immunother Cancer, 5:18 (2017)). The geometric mean across relevant genes listed in the table below was calculated to generate the composite score for each immune subset signature, namely CD45, B cells, CD8 T cells, cytotoxic cells, exhausted CD8, immature dendritic cells (iDC), macrophages, mast cells, neutrophils, NK CD56 dim cells, T cells, and regulatory T cells (Treg). The score was mean-centered and standardized in all analyses.

TABLE 3
Relevant genes expressed by immune cell
populations in mTIL signatures.

| mTIL signature cell type | Gene list |
|---|---|
| CD45 | PTPRC |
| B cells | MS4A1, TNFRSF17, KIAA0125 |
| CD8 T cells | CD8A, FLT3LG |
| Cytotoxic cells | GNLY, GZMA, GZMB, KLRB1, |
| | KLRK1, NKG7 |
| Exhausted CD8 | LAG3, PTGER4 |
| iDC | CARD9, F13A1 |
| Macrophages | CD163, CD68, CD84, MS4A4A |
| Mast cells | TPSAB1, CPA3, TPSB2 |
| Neutrophils | CSF3R, FCGR3B |
| NK CD56dim cells | IL21R |
| T cells | CD3D, CD3E, CD3G, CD6, TRAT1 |
| Regulatory T cells | FOXP3 |
| Adaptive immune | CD200R1, CD226, TRAF6, CTLA4, |
| | CD3G, CDH1, ICOS, ITGAL, MAP3K8, |
| | CD28, NFKBIA, ICAM3, CSK, HRAS, |
| | SELL, PDPK1, C3, PIK3CD, CD3E, |
| | ZAP70, FYN, KLRK1, NCF4, CD3D, |
| | PTPRC, AMICA1, CD4, LCP2, |
| | TNFRSF14, FYB, FOXO1, HLA-DMB, |
| | CD96, CD74, ITGB2, HLA-DMA, LCK, |
| | FCGR2B, and HCST |

### Statistical Analysis

For outcome analysis, recurrence-free survival (RFS) was defined as the time from random assignment to breast cancer recurrence (local, regional, or distant recurrence of breast cancer or breast cancer-related death). The time to event for patients who died without recurrence was considered censored at the time of death. Cox proportional hazard models were used to generate point estimates of hazard ratios (HRs) and corresponding 95% confidence intervals (CIs) to assess the benefit of trastuzumab for RFS and overall survival (OS) comparisons.

### Results

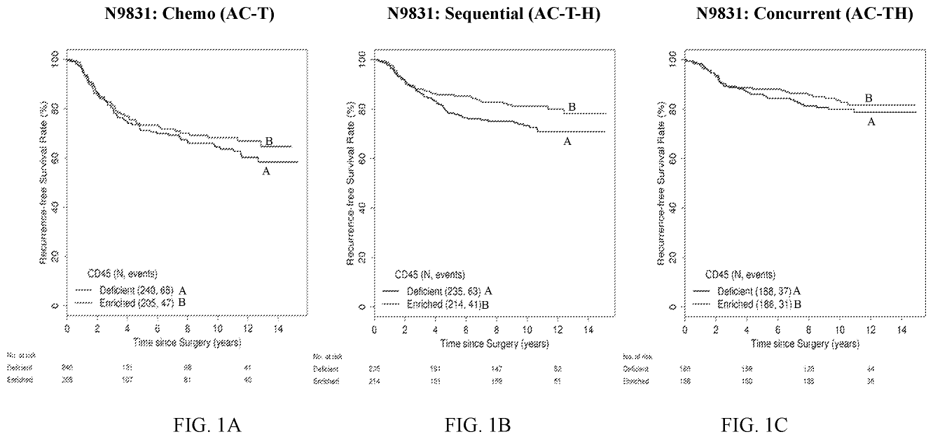

Enrichment of total mTIL measured by CD45 was significantly associated with improved RFS in patients receiving chemotherapy alone. Among patients who received trastuzumab, enrichment of total mTIL CD45 was not significantly associated with RFS in patients who received concurrent trastuzumab. (FIG. 1). However, improved RFS was observed in patients with enriched total mTIL CD45 who received sequential trastuzumab. The 10-year Kaplan-Meier estimates for RFS in sequential trastuzumab arm patients with CD45 enrichment or no enrichment were 81.3% and 72.6%, respectively (HR 0.63, 95% CI, 0.42-0.93; p=0.02), and in arm C were 83.6% and 79.8%, respectively (HR 0.79, 95% CI 0.49-1.28; p=0.34). In patients with CD45 mTIL deficient, improved RFS was observed when trastuzumab was given concurrently with chemotherapy.

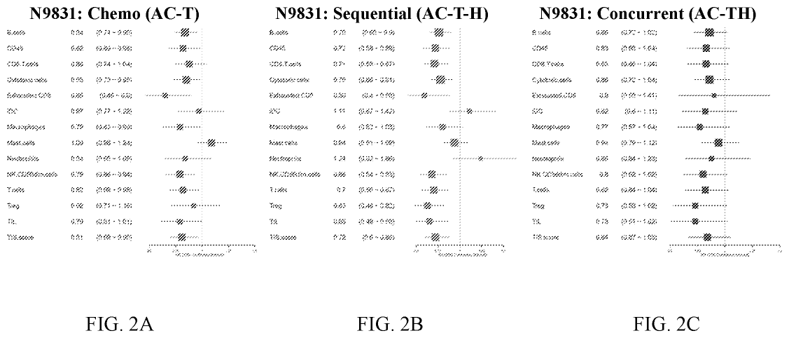

Besides total CD45 mTIL signature, 11 additional immune subset signatures were also evaluated in all treatment arms. None of the immune subset signature was significantly associated with outcome in patients receiving concurrent trastuzumab (FIG. 2C). However, several immune subset signatures were significantly associated with improved RFS in patients receiving chemotherapy alone, including B cells, cytotoxic cells, exhausted CD8, macrophages, NK CD56 dim cells, T cells, and Treg (FIG. 2A). Among patients who received sequential trastuzumab, several immune subset signatures are also associated with improved RFS, including B cells, CD8 T cells, cytotoxic cells, exhausted CD8, NK CD56 dim cells, T cells, and Treg (FIG. 2B).

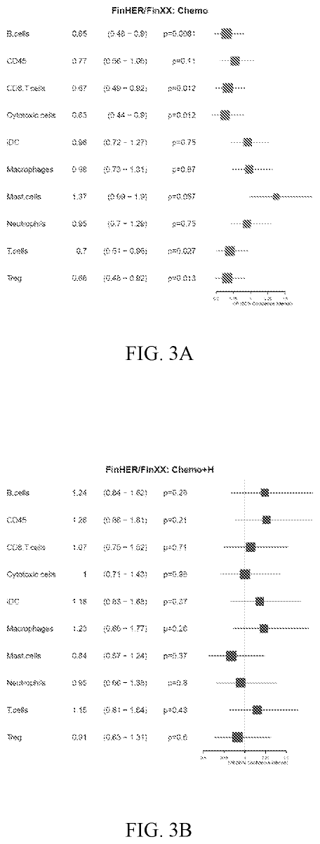

Similar trends were also observed in a separate data set from the combined FinHer and FinXX trials, in which, none of mTIL signatures were significantly associated with outcome among patients who received concurrent trastuzumab (FIG. 3). Enrichments of immune subset signatures, including B-cell, CD8-T-cells, cytotoxic-cell, T-cells, and regulatory-T-cell signatures were significantly associated with improved RFS in patients who received chemotherapy alone. However, in the FinHer and FinXX trials, total CD45 mTIL signature was not significantly associated with RFS in chemotherapy alone arm.

Contribution from each immune related genes and outcome in patients treated with sequential trastuzumab was evaluated. Using Gene Ontology and Gene Set Enrichment analysis of immune related genes significantly associated with improved outcome in patients treated with concurrent trastuzumab, adaptive immune reactome was identified and adaptive immune signature (AIS) was developed. Enrichment of this 17 gene AIS (i.e., (CD200R1, CD226, TRAF6, CTLA4, CD3G, CDH1, ICOS, ITGAL, MAP3K8, CD28, NFKBIA, ICAM3, CSK, HRAS, SELL, PDPK1, and CD3)).was found to be significantly associated with improved RFS in patients who received concurrent trastuzumab (AC-TH). Further validation was performed in chemotherapy only and sequential trastuzumab arms of the NCCTG with similar associations observed in sequential arm and chemotherapy only arm.

Patients in both AIS deficient and enriched groups appear to benefit from trastuzumab, particularly when given as a concurrent fashion. However, comparing between AIS deficient and AIS enriched patients, patients with enrichment of this signature was significantly associated with higher baseline sTIL (p<0.0001), ER negativity (p=0.047), and larger tumor size (p<0.0001) but not lymph node status or tumor grade (Table 4).

TABLE 4
Baseline characteristics among patients with enriched vs.
deficient in adaptive immune signature in the NCCTG N9831.

| | Deficient | Enriched | | |
|---|---|---|---|---|
| | AIS | AIS | Total | |
| | (N = | (N = | (N = | |
| Characteristics | 689) | 689) | 1378) | p value |
| Age | | | | 0.0109 |
| Mean | 49.4 | 50.6 | 50.0 | |
| Median | 49.0 | 51.0 | 50.0 | |
| Range | (22.0-80.0) | (23.0-79.0) | (22.0-80.0) | |
| Tumor Size | | | | <0.0001 |
| Mean | 3.0 | 2.7 | 2.9 | |
| Median | 2.5 | 2.3 | 2.5 | |
| Range | (0.1-13.2) | (0.1-15.0) | (0.1-15.0) | |
| Nodal Status | | | | 0.2907 |
| N0 | 132 (19.2%) | 134 (19.4%) | 266 (19.3%) | |
| N1 | 274 (39.8%) | 274 (39.8%) | 548 (39.8%) | |
| N2 | 198 (28.7%) | 175 (25.4%) | 373 (27.1%) | |
| N3 | 85 (12.3%) | 106 (15.4%) | 191 (13.9%) | |
| Menopausal Status | | | | 0.1059 |
| Premenopausal | 370 (53.7%) | 340 (49.3%) | 710 (51.5%) | |
| Postmenopausal | 319 (46.3%) | 349 (50.7%) | 668 (48.5%) | |
| Grade | | | | 0.6838 |
| Missing | 8 | 9 | 17 | |
| Low (1-2) | 192 (28.2%) | 185 (27.2%) | 377 (27.7%) | |
| High (3) | 489 (71.8%) | 495 (72.8%) | 984 (72.3%) | |
| Hormonal Status | | | | 0.5527 |
| Negative | 319 (46.3%) | 330 (47.9%) | 649 (47.1%) | |
| Positive | 370 (53.7%) | 359 (52.1%) | 729 (52.9%) | |
| Treatment Arm | | | | 0.0448 |
| A | 261 (37.9%) | 219 (31.8%) | 480 (34.8%) | |
| B | 226 (32.8%) | 260 (37.7%) | 486 (35.3%) | |
| C | 202 (29.3%) | 210 (30.5%) | 412 (29.9%) | |

In NeoALTTO, 134 out of 244 (54.9%) patients were considered deficient in the AIS. NeoALTTO patients with deficient AIS had significantly lower pathologic complete response (pCR) rate, compared to patients with enriched AIS (Chi-squared p<0.0001) (Table 5). In patients who received trastuzumab (arm B), pCR was observed in 43% of patients with enriched AIS compared to 8% in patients with deficient AIS (OR=9.12, 2.25-54.42, p=0.0004). pCR was observed in 17% of patients with deficient AIS in patients who received lapatinib (arm A) and in 52% in patients who received the combination of trastuzumab and lapatinib (arm C). Among AIS deficient patients, observed 6 year-EFS was longer in those treated with lapatinib (68%, 95% CI 0.55-0.85) or the combination of trastuzumab and lapatinib (73%, 95% CI 0.60-0.88), compared to trastuzumab alone, (63%, 95% CI 0.49-0.80).

TABLE 5
Complete pathological response rate (% pCR) and %6-year Event Free
Survival rate in the NeoALTTO trial based on treatment arms and
adaptive immune signature status (enriched vs. deficient).

| | % pCR | | %6-yr EFS | |
|---|---|---|---|---|
| Treatment Arms | AIS deficient | AIS enrich | AIS deficient | AIS enrich |
| Lapatinib (arm A) | 17% | 21% | 68% | 65% |
| | | | 95% CI 0.55-0.85 | 95% CI 0.51-0.84 |
| Trastuzumab (arm B) | 8% | 43% | 63% | 74% |
| | | | 95% CI 0.49-0.80 | 95% CI 0.61-0.90 |
| Combination of | 52% | 44% | 73% | 85% |
| trastuzumab and | | | 95% CI 0.60-0.88 | 95% CI 0.74-0.98 |
| lapatinib (arm C) | | | | |

Collectively, the results provided herein demonstrate that pre-existing adaptive immune enrichment may play a significant role in determining outcome in patients having HER2-positive cancer receiving trastuzumab.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having a human epidermal growth factor receptor 2 (HER2)-positive cancer and having an enriched adaptive immune signature, wherein said method comprises:
administrating trastuzumab to said mammal, and concurrently administering one or more chemotherapeutic agents to said mammal.

2. The method of claim 1, wherein said enriched adaptive immune signature comprises an elevated level of polypeptides expressed by CD200 receptor 1 (CD200R1), cluster of differentiation (CD) 226 (CD226), TNF Receptor Associated Factor 6 (TRAF6), cytotoxic T-lymphocyte associated protein 4 (CTLA4), CD3-gamma (CD3G), cadherin-1 (CDH1), inducible T-cell co-stimulator (ICOS), integrin alpha L (ITGAL), mitogen-activated protein kinase kinase kinase 8 (MAP3K8), CD28, NFKB inhibitor alpha (NFKBIA), intercellular adhesion molecule 3 (ICAM3), C-terminal Src kinase (CSK), HRAS, SELL, 3-phosphoinositide-dependent protein kinase 1 (PDPK1), and CD3.

3. The method of claim 1, wherein said enriched adaptive immune signature comprises an elevated level of polypeptides expressed by CD200 receptor 1 (CD200R1), cluster of differentiation (CD) 226 (CD226), TNF Receptor Associated Factor 6 (TRAF6), cytotoxic T-lymphocyte associated protein 4 (CTLA4), CD3-gamma (CD3G), cadherin-1 (CDH1), inducible T-cell co-stimulator (ICOS), integrin alpha L (ITGAL), mitogen-activated protein kinase kinase kinase 8 (MAP3K8), CD28, NFKB inhibitor alpha (NFKBIA), intercellular adhesion molecule 3 (ICAM3), C-terminal Src kinase (CSK), HRAS, SELL, 3-phosphoinositide-dependent protein kinase 1 (PDPK1), CD3, phosphoinositide-3-kinase catalytic delta (PIK3CD), CD3-epsilon (CD3E), 70 kDa zeta-associated protein (ZAP70), FYN, killer cell lectin like receptor K1 (KLRK1), neutrophil cytosolic factor 4 (NCF4), CD3 delta (CD3D), Protein tyrosine phosphatase receptor type C (PTPRC), adhesion molecule interacts with CXADR antigen 1 (AMICA1), CD4, lymphocyte cytosolic protein 2 (LCP2), TNF receptor superfamily member 14 (TNFRSF14), FYN-T-binding protein (FYB), forkhead box protein O1 (FOXO1), HLA class II histocompatibility antigen DM beta (HLA-DMB), CD96, CD74, integrin beta chain-2 (ITGB2), HLA class II histocompatibility antigen DM alpha (HLA-DMA), LCK, Fc fragment of IgG receptor IIb (FCGR2B), and hematopoietic cell signal transducer (HCST).

4. The method of claim 1, wherein said administering one or more chemotherapeutic agents to said mammal comprises first administering 3 cycles of docetaxel and capecitabine, and subsequently administering 3 cycles of cyclophosphamide, epirubicin, and capecitabine.

5. The method of claim 1, wherein said administering one or more chemotherapeutic agents to said mammal comprises first administering 3 cycles of docetaxel, and subsequently administering by 3 cycles of cyclophosphamide, epirubicin, and fluorouracil.

6. A method for treating a mammal having a human epidermal growth factor receptor 2 (HER2)-positive cancer and having a deficient adaptive immune signature, wherein said method comprises:
administering trastuzumab to said mammal, and sequentially administering one or more chemotherapeutic agents to said mammal.

7. The method of claim 6, wherein said administering one or more chemotherapeutic agents to said mammal comprises first administering 3 cycles of docetaxel and capecitabine, and subsequently administering 3 cycles of cyclophosphamide, epirubicin, and capecitabine.

8. The method of claim 6, wherein said administering one or more chemotherapeutic agents to said mammal comprises first administering 3 cycles of docetaxel, and subsequently administering by 3 cycles of cyclophosphamide, epirubicin, and fluorouracil.

9. The method of claim 6, wherein said sequentially administering said one or more chemotherapeutic agents comprises administering said one or more chemotherapeutic agents after administering said trastuzumab.

10. The method of claim 6, wherein said sequentially administering said one or more chemotherapeutic agents comprises administering said one or more chemotherapeutic agents before administering said trastuzumab.

11-14. (canceled)

15. A method for treating a mammal having a human epidermal growth factor receptor 2 (HER2)-positive cancer and having an enriched molecular tumor infiltrating lymphocyte (mTIL) signature, wherein said method comprises:
administering trastuzumab to said mammal, and sequentially administering one or more chemotherapeutic agents to said mammal.

16. The method of claim 15, wherein said enriched mTIL signature comprises an increased abundance of B cells, CD8 T cells, cytotoxic cells, exhausted CD8, immature dendritic cells, macrophages, mast cells, neutrophils, NK CD56 dim cells, T cells, and regulatory T cells.

17. The method of claim 15, wherein said administering one or more chemotherapeutic agents to said mammal comprises first administering 3 cycles of docetaxel and capecitabine, and subsequently administering 3 cycles of cyclophosphamide, epirubicin, and capecitabine.

18. The method of claim 15, wherein said administering one or more chemotherapeutic agents to said mammal comprises first administering 3 cycles of docetaxel, and subsequently administering by 3 cycles of cyclophosphamide, epirubicin, and fluorouracil.

19. The method of claim 15, wherein said sequentially administering said one or more chemotherapeutic agents comprises administering said one or more chemotherapeutic agents after administering said trastuzumab.

20. The method of claim 15, wherein said sequentially administering said one or more chemotherapeutic agents comprises administering said one or more chemotherapeutic agents before administering said trastuzumab.

21-24. (canceled)

25. The method of claim 1, wherein said mammal is a human.

26. The method of claim 1, wherein said cancer is breast cancer.

27. The method of claim 26, wherein said breast cancer is a hormone receptor positive breast cancer.
